(19) United States
(12) Patent Application Publication
(10) Pub. No.: US 2011/0212047 A1
(43) Pub. Date: Sep. 1, 2011

(54) Polymer Therapy for the Treatment of Chronic Microvascular Diseases

(75) Inventors: Robert L. Hunter, Bellaire, TX (US); Martin Emanuele, San Diego, CA (US)

(73) Assignee: Synth Rx, Inc., Bellaire, TX (US)

(21) Appl. No.: 12/672,907

(22) PCT Filed: Aug. 11, 2008
(86) PCT No.: PCT/US08/09603

§ 371 Date: Feb. 10, 2010

Related U.S. Application Data
(60) Provisional application No. 60/955,046, filed on Aug. 10, 2007.

(51) Int. Cl. A61K 31/765; A61P 9/00; A61P 9/04; A61P 9/10; A61P 27/16; A61P 27/02; A61P 29/00; A61P 7/02; C12Q 1/44; A61B 5/145

(52) U.S. Cl. 424/78.17; 435/19; 600/309

(57) ABSTRACT

Methods are provided for the treatment of chronic microvascular diseases characterized by inflammation, such as age-related macular degeneration, by administering a polyoxyethylene/polyoxypropylene copolymer. Although a single dose can be effective, multiple treatments can be administered to achieve an optimal and sustained effect. Preferably, a single administration followed by repeated weekly administration of the pharmaceutical composition, but not continuous infusion, achieves a desired effect. Methods of diagnosis and characterization of chronic microvascular diseases using the copolymer are also provided.

### TECHNICAL FIELD

This application relates to the use of polyoxyethylene/polyoxypropylene copolymers for treating, for inhibiting/preventing progression of, or for diagnosing chronic microvascular diseases characterized by inflammation, such as macular degeneration.

### BACKGROUND

Inflammation has emerged as a primary pathogenic mechanism that links microvascular dysfunction and injury with several chronic diseases, including age-related macular degeneration (AMD), congestive heart failure, cerebral ischemia, transient ischemic attacks, diabetic retinopathy, diabetic peripheral vascular disease, peripheral vascular disease, and sudden hearing loss. The inflammation in these diseases is observed using standard indicators of inflammation, such as elevated baseline white blood cell count, erythrocyte sedimentation rate, elevated C-reactive protein, and homocysteine, and may be initiated by activators such as phospholipase-A2 (PLA2), VEGF, IL-1, IL-6, Factor B, and fibrinogen. Areas of the microvasculature that are not associated with overt lesion development also assume the inflammatory phenotype characterized by oxidative stress and endothelial cell activation.

The clinical manifestations of microvascular inflammatory disease depend on the severity of lesions in a particular site, the metabolic needs of the site, and its physiologic function. For example, a lesion severe enough to produce tiny hemorrhages would cause only inconsequential blemishes in the skin whereas the same lesion in the eye could produce blindness.

Angiogenesis occurs in diverse physiological conditions such as normal growth and wound healing as well as in pathological conditions such as age-related macular degeneration, diabetic retinopathy, and cancer. Inflammatory cells, namely monocytes/macrophages, T lymphocytes and neutrophils, participate in the angiogenic process by secreting growth factors and cytokines that may affect endothelial cell (EC) functions, including EC proliferation, migration, and activation. The new vessels formed as a result of the inflammation may fail to contain proteins in the same way as normal vessels and thereby contribute to tissue edema. Development of abnormal blood vessels is a major problem in several retinal diseases. Endothelial dysfunction and inflammation have been hypothesized as key mechanisms that contribute to the development of retinal microvascular changes in patients with age-related macular degeneration, diabetes, and other disorders.

Age-related macular degeneration, or AMD, is a chronic and progressive disease of the most sensitive part of the eye, the macula, that results in the loss of central vision. The most common symptoms include central distortion, loss of contrast sensitivity, and loss of color vision, none of which can be corrected by glasses, contact lenses, or laser surgery. People with age-related macular degeneration often have difficulty living independently and performing routine daily activities. Approximately 15 million people in the United States suffer from age-related macular degeneration.

Age-related macular degeneration is the leading cause of late onset visual impairment and legal blindness in people over the age of 50 in the industrialized world. According to the U.S. Census Bureau, the number of people in the United States aged 50 or older is approximately 80 million and is expected to increase by approximately 40% over the next two decades. It is expected that this rise in the population of elderly individuals will result in a significant increase in the number of cases of age-related macular degeneration in the United States.

The precise cause of age-related macular degeneration is not known and is probably multifactorial. Genetic, environmental and life style factors have been identified. There are two forms of age-related macular degeneration. Wet age-related macular degeneration afflicts 10-15% of patients, whereas dry age-related macular degeneration affects the remainder. Dry age-related macular degeneration occurs when the retina becomes atrophic, waste material (drusen) accumulates, and light-sensitive cells in the macula slowly break down, gradually blurring central vision in the affected eye. Dry age-related macular degeneration is thought to result from impaired microcirculation and inflammation in the retina of the eyes.

Patients with dry age-related macular degeneration are diagnosed into National Eye Institute Categories 1-4, depending on their risk of progression, or conversion, to wet age-related macular degeneration within five years (AREDS Report No. 14. Arch. Ophthalmol. (2005), 123:1207-1214):

Category 1—no macular abnormality in either eye to a few small drusen;

Category 2—many small or a few intermediate drusen, or pigment abnormalities;

Category 3—at least one large drusen, extensive intermediate drusen, or noncentral geographic atrophy;

Category 4—advanced AMD or lesions of AMD with visual acuity of less than 20/32 in 1 eye.

Currently, there is no FDA-approved therapy for dry age-related macular degeneration. A high-dose formulation of antioxidants and zinc has been reported to slow progression of dry age-related macular degeneration in about 25% of patients, but not to restore vision already lost from undergone clinical trials in the U.S. Rheopheresis has improved vision in some dry age-related macular degeneration patients with early advanced disease (Pullido, J. S., et al., Preliminary analysis of the final multicenter investigation of Rheophoresis for age related macular degenertion (AMD) trial (MIRA-1) results, Trans. Am. Ophthalmol. Soc. 104: 221-231 (2006)). The proteins and lipids removed from the blood by rheopheresis regenerate in a period of days. Nevertheless, improvements following a course of eight half-day treatments over a period of ten weeks may last for a year. Unfortunately, once dry age-related macular degeneration reaches the advanced stage, no treatment is available for the prevention of vision loss.

Wet age-related macular degeneration occurs when abnormal blood vessels behind the retina start to grow under the macula and leak blood and fluid, causing damage to the macula. Abnormal production of vascular endothelial growth factor (VEGF) contributes to the pathology. If treatment to control development of abnormal blood vessels is not received soon enough, the damage becomes irreversible. Wet age-related macular degeneration can be treated by destroying or inhibiting the development of new blood vessels with laser surgery, photodynamic therapy, and/or injections of drugs or immunologicals (e.g., anti-VEGF) that inhibit blood vessel formation into the eye. Unfortunately, the number of wet age-related macular degeneration patients that are treatable with current therapy is relatively small. The microvascular inflammatory disease that originally produced atrophy of the retina and stimulated the production of abnormal vessels persists. Consequently, the loss of vision may progress despite treatment.

### Diabetic Retinopathy

Diabetic retinopathy is the most common diabetic eye disease and is a leading cause of blindness in American adults. All people with diabetes, both type 1 and type 2, are at risk. Between 40 to 45 percent of Americans diagnosed with diabetes have some stage of diabetic retinopathy. Diabetic retinopathy has four stages: mild nonproliferative retinopathy, moderate nonproliferative retinopathy, severe nonproliferative retinopathy, and proliferative retinopathy.

Diabetic retinopathy is an inflammatory disease with severe microvascular impairment. Blood vessels damaged from diabetic retinopathy can cause vision loss in two ways. Fragile, abnormal blood vessels can develop that leak blood into the center of the eye, blurring vision. This is called “proliferative retinopathy,” the fourth and most advanced stage of the disease. In addition, fluid can leak into the center of the macula, making the macula swell and blurring vision. This condition is called “macular edema,” which can occur at any stage of diabetic retinopathy, although it is more likely to occur as the disease progresses.

untreated, proliferative retinopathy can cause severe vision loss and even blindness. Macular edema may be treated with laser surgery referred to as focal laser treatment. Once patients develop proliferative retinopathy, they always will be at risk for new bleeding that impairs sight and can never be cured. Recent studies with immunologics that block formation of abnormal blood vessels have shown promise in slowing this disease.

### Other Chronic Microvascular Diseases

Inflammation has also been found to be associated with other chronic diseases involving microvasculature disorders. A microcirculatory disorder of the inner ear may be the final common pathway of a variety of sensorineural hearing loss disorders including sudden sensorineural hearing loss. Rest pain and trophic changes associated with Critical Limb Ischemia (CLI), the most advanced stage of Peripheral Arterial Disease (PAD), are predominantly related to a critical reduction in peripheral microcirculation. In addition, low-grade inflammation contributes to human heart failure. Further, inflammatory markers may reflect not only peripheral disease, but also cerebral disease mechanisms related to dementia.

As inflammatory markers associated with many chronic microvascular diseases are often measurable long before clinical symptoms appear, there is a great need for methods of diagnosing and treating chronic microvascular diseases upon early detection of these high risk factors.

### SUMMARY

Methods for inhibiting/preventing progression of and for treating chronic microvascular diseases characterized by inflammation are described herein. In accordance with the described methods, a pharmaceutical composition containing a polyoxyethylene/polyoxypropylene linear copolymer is administered to a patient suffering from the chronic microvascular disease. A single administration of the pharmaceutical composition achieves a desired effect. Subsequent administrations may be necessary to produce an optimal and sustained effect.

The polyoxyethylene/polyoxypropylene copolymer in the pharmaceutical composition administered in the methods described herein has the following chemical formula:

HO(C₂H₄O)ₐ—(C₃H₆O)_{b}—(C₂H₄O)ₐH

wherein b is an integer such that the hydrophobe represented by (C₃H₆O)_{b} (i.e., the polyoxypropylene portion of the copolymer) has a molecular weight of approximately 950 to 4000 daltons, preferably about 1200 to 3500 daltons, and a is an integer such that the hydrophile portion represented by (C₂H₄O)ₐ (i.e., the polyoxyethylene portion of the copolymer) constitutes weight between 5,000 and 15,000 daltons.

A preferred copolymer is Poloxamer 188 having the following chemical formula:

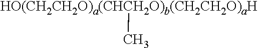

wherein the molecular weight of the hydrophobe (C₃H₆O)_{b} (the polyoxypropylene portion of the copolymer), is approximately 1750 daltons and the total molecular weight of the compound is approximately 8400 daltons.

A further preferred copolymer is purified Poloxamer 188 with reduced low and high molecular weight contaminants, wherein the polydispersity value of the polyoxypropylene/polyoxyethylene block copolymer is less than approximately 1.07 or less than 1.05 or less than 1.03 as described in U.S. Pat. No. 5,696,298 that is incorporated by reference herein.

Suitable microvascular diseases characterized by chronic inflammation to be treated with the method described herein include, but are not limited to, age-related macular degeneration (AMD), congestive heart failure, cerebral ischemia, transient ischemic attacks, diabetic retinopathy, diabetic peripheral vascular disease, peripheral vascular or arterial disease, critical limb ischemia, and sudden sensorineural hearing loss.

Also provided are methods of diagnosing or characterizing patients having chronic microvascular diseases by administering the polyoxyethylene/polyoxypropylene copolymer and monitoring changes in inflammatory indicators, blood flow, and/or tissue oxygenation induced by the polyoxyethylene/polyoxypropylene copolymer.

Accordingly, it is an object of the present invention to provide a method for treating microvascular diseases that are chronic and to restore function of the affected organ.

It is yet another object of the present invention to provide a method for treating chronic microvascular diseases to reduce the risk of progression to untreatable conditions.

It is yet another object of the present invention to provide a method for diagnosing or characterizing chronic microvascular diseases at an early stage of disease, preferably before symptoms even appear, so that more specific and effective treatment can be implemented and permanent damage avoided.

These and other objects, features and advantages of the present invention will become apparent after a review of the following detailed description of the disclosed embodiments and the appended claims.

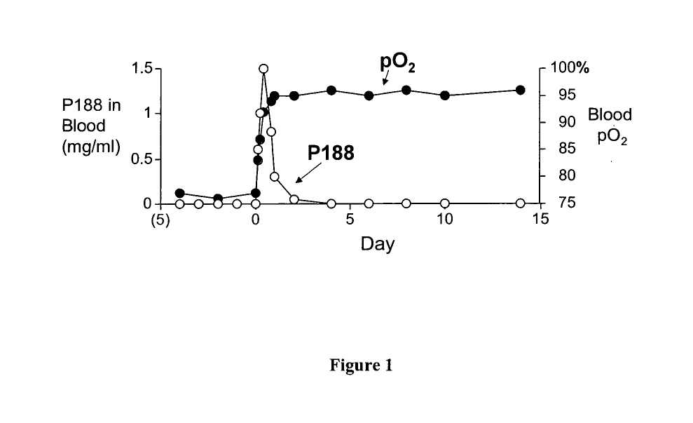

FIG. 1 is a graph of blood levels of P188 and oxygen (pO₂) in a child with acute chest syndrome of sickle cell disease versus time showing a prolonged improved blood oxygen level following a single infusion of 300 mg/kg P188 over eight hours.

### DETAILED DESCRIPTION

Methods for inhibiting/preventing progression of and for treating chronic microvascular diseases characterized by inflammation are provided. In accordance with the methods, a pharmaceutical composition containing a polyoxyethylene/polyoxypropylene copolymer is administered to a patient suffering from the chronic microvascular disease. Although a single dose may be effective, multiple treatments may be necessary to achieve an optimal and sustained effect. Preferably, a single administration or series of administrations achieves a desired effect that is sustained by repeated weekly, monthly, or less frequent administrations of the pharmaceutical composition.

The polyoxyethylene/polyoxypropylene copolymer in the pharmaceutical composition administered in the methods described herein is a linear copolymer having the following chemical formula:

HO(C₂H₄O)ₐ—(C₃H₆O)_{b}—(C₂H₄O)ₐH

wherein b is an integer such that the hydrophobe represented by (C₃H₆O)_{b} has a molecular weight of approximately 950 to 4000 daltons, preferably about 1200 to 3500 daltons, and a is an integer such that the hydrophile portion represented by (C₂H₄O)ₐ constitutes approximately 50% to 95% by weight of the compound. The copolymer has a preferred molecular weight between 5,000 and 15,000 daltons.

The polyoxyethylene/polyoxypropylene copolymer is a surface-active agent, or surfactant, and is formed by ethylene oxide-propylene oxide condensation. The copolymer is a triblock copolymer of the form poly(ethylene oxide)-poly(propylene oxide)-poly(ethylene oxide).

A preferred copolymer for use in the methods described herein is Poloxamer 188 (P188, commercially available from BASF, Florham Park, N.J. as Pluronic® F68) having the following chemical formula:

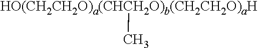

wherein the molecular weight of the hydrophobe (C₃H₆O)_{b} is approximately 1750 daltons and the total molecular weight of the compound is approximately 8400 daltons. P188 has a molecular poly(ethylene oxide) weight ratio of 4:2:4.

Another preferred copolymer is a purified Poloxamer 188 having reduced low and high molecular weight contaminants and a polydispersity less than 1.07, 1.05, or 1.03.

Also provided are methods of diagnosing or characterizing patients having chronic microvascular diseases by monitoring polyoxyethylene/polyoxypropylene copolymer-induced changes in inflammation as measured by standard inflammatory indicators.

Suitable chronic disease conditions to be treated or diagnosed with the methods described herein are those are characterized by microvascular inflammation, particularly microvascular inflammation affecting endothelial cells, blood cells, and involving inflammatory mediators such as fibrinogen, VEGF, tissue factor, TPA, IL1 CRP, IL-8, and phospholipase A2. These chronic diseases include age-related macular degeneration (AMD), congestive heart failure, cerebral ischemia, transient ischemic attacks, critical limb ischemia, diabetic retinopathy, diabetic peripheral vascular disease, peripheral vascular or arterial disease, and sudden sensorineural hearing loss. The surprisingly prolonged effect of polyoxyethylene/polyoxypropylene linear copolymers, such as Poloxamer 188, on these chronic disease conditions persists for weeks to months to years depending on the conditions and may be achieved with administration of a single administration or a series of administrations. The composition may be conveniently administered by intravenous infusion, but other routes of injection are also useful.

### DEFINITIONS

The terms “a”, “an”, and “the” as used herein are defined to mean one or more and include the plural unless the context is inappropriate.

The phrase “consisting essentially of is used herein to exclude any elements that would substantially alter the essential properties of the composition to which the phrase refers. Thus, the description of a pharmaceutical composition “consisting essentially of . . . ” excludes any chemicals or other ingredients that would substantially alter the activity of that composition.

The term “effective amount” refers to the amount of the composition which, when administered to a human or animal, elicits an anti-inflammatory response; prevents, reduces, or lessens angiogenesis; causes a reduction in vascular proliferation; and/or inhibits pathogenic vascular disease. The effective amount is readily determined by one of skill in the art following routine procedures. Specific effective dosages and dosage schedules for administering the polyoxyethylene/polyoxypropylene copolymers according to the methods described herein may be determined empirically or by other approaches; making such determinations is routine to those of ordinary skill in the art. The skilled artisan will understand that the dosage of route of administration, the particular copolymer to be used, other drugs being administered, and the age, condition, sex, and extent of the disease in the subject. The dosage can be adjusted by the individual physician in the event of any counterindications.

For example, pharmaceutically-acceptable copolymer compositions can be administered intravenously, intramuscularly, parenterally, subcutaneously, or via inhalation as an aerosol in a range of approximately 20 mg/kg to 500 mg/kg patient (e.g., but not limited to, about: 20-50, 20-100, 20-250, 50-100, 50-200, 50-300, 100-200, 100-300, 100-400, 250-450; 300-475 mg/kg, etc., e.g., but not limited to, about 20, 50, 75, 100, 150, 200, 250, 300, 350, 400, 450, 475, 500, etc. mg/kg patient) though this range is not intended to be limiting. Preferably, the concentration of copolymer in the solution being administered is approximately 5% to 25% (e.g., but not limited to, about 5-10, 5-15, 5-20, 10-15, 10-20%, etc., e.g., about 5, 6, 7, 8, 9, 10, 11, 12, 13, 14, 15, 16, 17, 18, 19, 20, 21, 22, 23, 24, 25%). The actual amount of the composition required to elicit the desired effect will vary for each individual patient, depending on the disease being treated and the response of the individual. The specific amount to be administered to an individual can be readily determined by one of ordinary skill in the art by techniques routinely used by such skilled artisans.

The efficacy of administration of a particular dose of polyoxyethylene/polyoxypropylene copolymer can be determined by evaluating the particular aspects of the medical history, signs, symptoms, and objective laboratory tests that are known to be useful in evaluating the status of a subject in need of treatment for a chronic microvascular disease as described herein. These signs, symptoms, and objective laboratory tests will vary, depending upon the particular disease or condition being treated or prevented, as will be known to any clinician who treats such patients or a researcher conducting experimentation in this field. For example, if, based on a comparison with an appropriate control group and knowledge of the normal progression of disease in the general population or the particular individual: 1) a patient's frequency or severity of recurrences is shown to be improved, 2) the progression of the disease is shown to be stabilized or delayed, or 3) the need for use of other medications for treating the condition or disease is lessened or obviated, then a particular treatment will be considered efficacious.

By “treat” is meant to administer a copolymer as described herein to a subject in order to: stabilize or delay the progression of a chronic microvascular disease or condition within the subject; decrease the frequency or severity of a sign or symptom and/or recurrences of a chronic microvascular disease or condition within the subject; and/or eliminate a chronic microvascular disease or condition within the subject.

administration of a polyoxyethylene/polyoxypropylene copolymer to a subject as described herein stabilizes and/or delays progression of a chronic microvascular disease within the subject; decreases the frequency or severity of a sign or symptom and/or recurrences of a chronic microvascular disease or condition within the subject; and/or eliminates a chronic microvascular disease or condition within the subject.

Examples that indicate a desired or therapeutic effect can include, but are not limited to: improvement of (or slowing, stopping, and/or preventing a deterioration of) retinal blood flow in a patient with macular degeneration; improvement of (or slowing the rate of loss of, or preventing the rate of loss of) visual function in a patient with macular degeneration; a decrease in the amount of subretinal fluid and/or a decrease in the amount of macular edema in a patient with macular degeneration; improvement of (or slowing and/or stopping further loss of) peripheral microvascular blood flow in a patient with peripheral arterial disease; improvement of (or stabilization of) limb circulation and/or skin blood flow in a patient with critical limb ischemia; a partial or incomplete inhibition of retinal blood vessel proliferation in a patient with proliferative diabetic retinopathy; a decrease in the incidence and/or severity of transient ischemic attacks in a patient with transient ischemic attacks; a slowing and/or stopping of the progression of congestive heart failure and/or angina in a patient with chronic congestive heart failure with unstable angina.

One of ordinary skill in the art will understand how to achieve a desired or therapeutic effect by administration of the polyoxyethylene/polyoxypropylene copolymers as described herein.

By “anti-VEGF compound” is meant a therapeutic compound that at least partially inhibits the angiogenic activity of vascular endothelial growth factor (VEGF). Examples of anti-VEGF compounds include, but are not limited to, pegaptanib (MACUGEN®, a pegylated anti-VEGF aptamer) and ranizumab (LUCENTIS®; an anti-VEGF monoclonal antibody fragment).

By “bodily fluid” is meant any fluid that comes from the body of a patient or subject, including, but not limited to, blood, serum, urine, saliva, sputum, and/or tears.

### Poloxamer 188

Certain polyoxyethylene/polyoxypropylene copolymers have been found to have beneficial biological effects on several acute diseases when administered to a human or animal. These activities have been described in U.S. Pat. Nos. 4,801,452, 4,837,014, 4,873,083, 4,879,109, 4,897,263, 4,937,070, 4,997,644, 5,017,370, 5,028,599, 5,030,448, 5,032,394, 5,039,520, 5,041,288, 5,047,236, 5,064,643, 5,071,649, 5,078,995, 5,080,894, 5,089,260, RE Applications PCT/US2005/034790, PCT/US2005/037157 and PCT/US2006/006862, all of which are incorporated herein by reference.

Poloxamer 188 (P188) is a polyoxyethylene/polyoxypropylene linear copolymer surface active agent that binds to hydrophobic areas developed on injured cells and denatured proteins thereby restoring hydration lattices. Such binding facilitates sealing of damaged membranes and aborts the cascade of inflammatory mediators that could destroy the cell. This polymer also inhibits hydrophobic adhesive interactions that cause deleterious aggregation of formed elements in the blood. Poloxamer 188's anti-adhesive and anti-inflammatory effects are exhibited by enhancing blood flow in damaged tissue by reducing friction, preventing adhesion and aggregation of formed elements in the blood, maintaining the deformability of red blood cells, non-adhesiveness of platelets and granulocytes, the normal viscosity of blood, reducing apoptosis, and by multiple markers of inflammation including VEGF, various chemokines, interleukins, and chemokines.

The non-ionic surfactant, Poloxamer P188, has been used for many years to treat conditions associated with acute vascular obstruction and tissue damage. However, due to its short duration of action, rapid excretion from the body, and short half life, a continuous infusion of Poloxamer 188 was thought to be necessary for a prolonged effect. Thus, Poloxamer 188 has been considered useful only for acute diseases in which the immediate cause of disease (such as, for example, heart attack, sickle cell crisis, hemorrhagic shock or thrombus) was of short duration.

Purified Poloxamer 188 is a synthetic, nonionic, block copolymer of ethylene oxide and propylene oxide that has been purified to reduce both high and low molecular weight polymeric impurities. The copolymer with an average molecular weight of 8500±1000 daltons is composed of a single chain (block) of hydrophobic polyoxypropylene, flanked by two chains (blocks) of hydrophilic polyoxyethylene, and has the following structural formula:

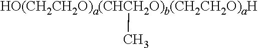

wherein the molecular weight of the hydrophobe (C₃H₆O)_{b} is approximately 1750 daltons and the total molecular weight of the compound is approximately 8400 daltons. P188 has a molecular weight of approximately 8400 g/mol and a poly(ethylene oxide)-poly(propylene oxide)-poly(ethylene oxide) weight ratio of 4:2:4.

Current clinical supplies of Poloxamer 188 are formulated as a clear, colorless, sterile, non-pyrogenic solution intended for intravenous (IV) administration with or without dilution. therapeutic effect. Each 100 ml vial contains 15 g of purified Poloxamer 188 (150 mg/ml), 308 mg sodium chloride USP, 238 mg sodium citrate USP, 36.6 mg citric acid USP and water for injection USP Qs to 100 ml. The pH of the solution is approximately 6.0 and has an osmolarity of 312 mOsm/L. These clinical formulations contain no bacteriostatic agents or preservatives.

### Methods of Treatment

Treatment of a patient having a chronic microvascular disease characterized by inflammation can be accomplished by administering to the patient a pharmaceutically acceptable composition containing a polyoxyethylene/polyoxypropylene copolymer, as described herein, at an effective dosage. Effective results may be obtained with a single dose. Multiple doses, such as weekly administration, but not continuous infusion, may be necessary to achieve optimal and sustained benefits.

Polyoxyethylene/polyoxypropylene linear copolymers, such as Poloxamer 188, are useful for the treatment of chronic microvascular disease by enhancing blood flow. Microvascular flow of blood in damaged tissue is compromised by multiple factors including inflammation, coagulation, decreased blood oxygen level, loss of deformability of red blood cells, sludging, viscosity, friction, endothelial cell damage, white cell adhesion and vascular tone. Poloxamer 188 has the ability to affect each of these parameters and significantly increase blood flow. Surprisingly, in certain conditions, the effect persists long after the copolymer has been cleared from the circulation.

Poloxamer 188 can also treat a chronic microvascular disease by inhibiting abnormal adhesion of blood cells to vascular endothelial cells. Such abnormal adhesion has been increasingly recognized as part of pathophysiology of multiple chronic microvascular diseases. Blocking such adhesion can break the cascade of events that sustains chronic inflammation.

Poloxamer 188 can be used as described herein to treat a chronic microvascular disease without exerting an immunosuppressive effect or potentiating infection. Being extensively used as a wound cleanser, Poloxamer 188 neither delays wound healing nor potentiates infection. It effectively inhibits deleterious inflammatory reactions associated with damaged tissue. Poloxamer 188 reduces tissue damage mediated by neutrophils by at least two mechanisms: (1) it inhibits the migration of neutrophils, chemotaxis and adhesion including to inflammatory loci, and (2) it inhibits the oxygen burst.

For the purpose of treating chronic microvascular diseases, Poloxamer 188 can be provided as substantially purified compositions as described above or placed in pharmaceutically acceptable formulations or delivered for sustained release using formulations and methods routes. In general, the compositions may be administered by various routes (e.g., intravenous, transdermal, intraperitoneal, intraspinal, intravitreal, subcutaneous or intramuscular). The preferred route is intravenous infusion.

Pharmaceutically acceptable carriers that can be combined with polyoxyethylene/polyoxypropylene copolymers for use in the methods of the invention are well-known in the art. For example, Remington: The Science and Practice of Pharmacy, 21^{st} Edition, Randy Hendrickson, Ed., Lippincott Williams & Wilkins, (2005), describes compositions and formulations suitable for pharmaceutical delivery of polyoxyethylene/polyoxypropylene copolymers as described herein. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically-neutral carriers, pharmaceutical compositions to be administered can contain minor amounts of non-toxic auxiliary substances, such as wetting or emulsifying agents, preservatives, and pH buffering agents and the like, for example sodium citrate.

The effective dosage of Poloxamer 188 and related polyoxyethylene/polyoxypropylene copolymers provided herein will depend on the disease state or condition being treated and other clinical factors such as weight and condition of the animal or human and the route of administration. Depending upon the half-life of Poloxamer 188 in the particular animal or human, the compound can be administered between several times per day to once a month or less. For example, the compound can be administered once per day, several times per week, once per week, twice per month, once per month, once every six to eight weeks, once every three months, etc. The methods described herein contemplate single as well as multiple administrations, given either concurrently or over an extended period of time.

It is to be understood that the methods provided herein have applications for both human and veterinary use.

The Poloxamer 188 formulations provided herein include those suitable for parenteral (including subcutaneous, intraperitoneal, intramuscular, intravenous, intravitreal, intradermal, intracranial, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s).

sterile injection solutions, which may contain anti-oxidants, buffers, bacteriostats and solutes that render the formulation isotonic with the blood of the intended recipient. The formulations may be presented in unit-dose or multi-dose containers, for example, sealed ampules and vials, and may be stored in a dried or freeze-dried (lyophilized) condition, requiring only the addition of the sterile liquid carrier, for example, water for injections, immediately prior to use.

Preferred unit dosage formulations are those containing a daily dose or unit, daily sub-dose, as recited above, or an appropriate fraction thereof, of the administered ingredient. It should be understood that, in addition to the ingredients particularly mentioned above, the formulations may include other agents conventionally used in the art having regard to the type of formulation in question. In addition, the pharmaceutical composition may contain other active ingredients such as, but not limited to, anti-VEGF, anti-inflammatories, antioxidants, free radical scavengers, and the like.

### Method of Diagnosis

Because the polyoxyethylene/polyoxypropylene copolymers described herein can selectively induce different responses by the same inflammatory indicators in patients having different chronic microvascular diseases, these different diseases can be diagnosed or characterized by administering the polyoxyethylene/polyoxypropylene copolymers, described herein, to patients identified as suffering from (or potentially suffering from) a chronic microvascular disease and then monitoring changes in inflammation by measuring inflammatory indicators and in tissue oxygenation by measuring hemoglobin saturation, e.g., using near-infrared spectroscopy. Observed changes in these inflammatory indicators are useful in identifying the inflammatory mediators involved in the diseases and devising suitable treatments for these diseases. For example, loss of vision in age related macular dystrophy may be due inflammation, death of light sensitive cells or other factors. The response to treatment measured by the parameters described above with the polyoxyethylene/polyoxypropylene copolymers described herein can help identify the underlying cause and thereby be of great value in determining the most appropriate therapy in a timely fashion.

The inflammatory indicators to be monitored include standard indicators such as, but not limited to, erythrocyte sedimentation rate (ESR), secretory phospholipase A₂, (sPLA₂), Factor B, fibrinogen, C-reactive protein (CRP), IL-1, IL-6, IL-8, and monocyte chemotractant protein-1 (MCP-1). For other examples of inflammatory indicators, see Shankar, A., et al. (2007), Association between circulating white blood cell count and long-term incidence of age-related macular degeneration: the Blue Mountains Eye Study. Am J Epidemiol 165:375-382; Vine, A. K., degeneration. Ophthalmology 112:2076-2080; Rattazzi, M., et al. (2003), C-reactive protein and interleukin-6 in vascular disease: culprits or passive bystanders? J Hypertens 21:1787-1803; Niessen, H. W., et al. (2003), Type II secretory phospholipase A2 in cardiovascular disease: a mediator in atherosclerosis and ischemic damage to cardiomyocytes? Cardiovasc Res 60:68-77; Devine, L., et al. (1996), Role of LFA-1, ICAM-1, VLA-4 and VCAM-1 in lymphocyte migration across retinal pigment epithelial monolayers in vitro. Immunology 88:456-462; Hill, T. A., et al. (1997), A new method for studying the selective adherence of blood lymphocytes to the microvasculature of human retina. Invest Ophthalmol Vis Sci 38:2608-2618; Bartosik-Psujek, H., et al. (2003), Markers of inflammation in cerebral ischemia. Neurol Sci 24:279-280.

The copolymer-induced differential responses by the same inflammatory marker in patients having similar inflammatory microvasculature is useful in understanding the cause of the disease, identifying the disease, and selecting the appropriate treatment for the disease. For example, the level of sPLA₂ has been shown to correlate with clinical severity of acute chest syndrome (ACS) and to be a differentiating factor among sickle cell patients with ACS and those with vaso-occlusive crisis or pneumonia. For example, measurement of a decrease in the level of sPLA₂ following Poloxamer 188 administration to a patient suffering from chronic microvascular disease provides valuable information as to the likely cause of the disease (acute chest syndrome) and, therefore, the selection of a treatment effective for that type of disease.

This invention is further illustrated by reference to the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

### Effect of Single Infusion of Poloxamer 188 on Chronic Inflammatory Disease of the Lung

A single infusion of Poloxamer 188 (P188, Burroughs-Welcome, Research Triangle Park, NC) provided a prolonged beneficial effect in treating a patient with chronic microvascular inflammatory disease.

Sickle cell disease is caused by a genetic abnormality in hemoglobin. However, many manifestations of the disease, especially acute chest syndrome are manifestations of microvascular inflammation.

conventional therapy with complete white out of the lungs that persisted in spite of vigorous treatment, including exchange transfusion and seven chest tubes and a blood oxygen (pO₂) level near 75% even with constant administration of oxygen. On day 21, he was infused with P188 in a bolus of 30mg/kg followed by 30 mg/kg/hr for eight hours.

As shown in FIG. 1, P188 was followed by rapid and sustained increase in oxygen saturation to near 95%. This blood oxygenation level was sustained for 14 days. In addition, he experienced no more episodes of desaturation or pneumothorax and better ventilator function as evidenced by decreased requirement for positive end expiratory pressure (PEEP). Unfortunately, the child subsequently died of multiple organ failure. Nevertheless, the prolonged beneficial effect of a single infusion of P188 on a chronic disease was completely unanticipated, especially because the underlying condition (crisis of sickle cell disease) persisted and worsened.

### EXAMPLE 2

### Anti-Inflammatory-Cytoprotective Activities of P188

Superior mesenteric artery occlusion (SMAO) in the rat produces ischemia/reperfusion injury (Gonzalez, E. A., et al., Conventional dose hypertonic saline provides optimal gut protection and limits remote organ injury after gut ischemia reperfusion. J. Trauma 61:66-73 (2006)). Rats were given SMAO for 60 minutes or similar sham surgery. Resuscitation was given 5 minutes prior to clamp removal and the animals were sacrificed at 6 hours. Table 1 shows the results of microarray analysis of gene expression in the small bowels of these animals. They show that the anti-inflammatory/cytoprotective effects of P188 extend to many mediators and markers of chronic microvascular inflammatory disease.

TABLE 1

| Markers of | | | | p |
|---|---|---|---|---|
| Inflammation | SHAM Control | SMAO | SMAO + P188 | value |
| VEGF | 91 ± 3 | 165 ± 53 | 109 ± 8 | <0.01 |
| CCL3 | 62 ± 6 | 414 ± 163 | 103 ± 25 | <0.01 |
| TLR2 | 120 ± 19 | 355 ± 176 | 145 ± 44 | <0.01 |
| IL1b | 450 ± 25 | 1427 ± 152 | 745 ± 57 | <0.01 |
| IL1R2 | 119 ± 10 | 1213 ± 443 | 301 ± 36 | <0.01 |
| IL6 | 1241 ± 3231 | 7009 ± 1241 | 3845 ± 138 | <0.01 |
| F3 (Tissue | 317 ± 35 | 1473 ± 130 | 1045 ± 233 | <0.01 |
| Factor) | | | | |
| TPA | 765 ± 116 | 4844 ± 865 | 2371 ± 333 | <0.01 |

### EXAMPLE 3

### Use of Poloxamer 188 to Treat Category 3 Age-Related Macular Degeneration

Poloxamer 188 is administered to a 75 year old man with non-exudative Category 3 Age-Related Macular Degeneration with multiple large soft drusen near the foveal center of his left eye. His eyes have a best spectacle corrected visual acuity (BSCVA) using the Early Treatment Diabetic Retinopathy Study (ETDRS) chart of 20/50 on the left and 20/32 on the right. He also has a score of 75 on the National Eye Institute Visual Functioning Questionnaire (NEI-VFQ). He is taking a high dose vitamin supplement recommended by the Age-Related Eye Disease Study (AREDS) of the National Eye Institute.

This patient is treated with an intravenous infusion of purified Poloxamer 188 at a constant rate over two hours to a total dose of 100 mg/kg body weight. This treatment is repeated weekly for 10 weeks.

Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L.

Eye examinations are conducted at months 1, 3, 6, 9 and 12 to measure improvement in BSCVA and NEI-VFQ. In addition, laser Doppler measurement of relative retinal and foveolar choroidal blood flow (ChBFlow) are obtained with laser Doppler flowmetry (LDF) (Oculix Instrument, Huntingdon Valley, Pa.) The results are expressed as AU (arbitrary units) that are standardized within technique (Metelitsina, T. I., et al. Investigative Ophthalmology & Visual Science, 49, 358-61, 2008). ChBFlow increased from 6.40 AU before treatment to 7.3 AU three hours after treatment. Subsequent measurements show ChBFlow consistently above the pre-treatment baseline.

### EXAMPLE 4

### Use of Poloxamer 188 to Treat Category 4 Age-Related Macular Degeneration

Poloxamer 188 is administered to a 70 year old woman with Category 4 Age-Related Macular Degeneration with multiple large soft drusen in the left eye and early wet age-related macular degeneration and in the right eye. Her eyes have BSCVA using the ETDRS chart of a high dose vitamin supplement recommended by AREDS.

This patient is treated with an intravenous infusion of purified Poloxamer 188 at a constant rate over two hours to a total dose of 100 mg/kg body weight. Improved retinal microvascular blood flow and foveolar choroidal blood flow (ChBFlow) is demonstrated by laser Dopplerocular examination immediately following treatment This treatment is repeated three times a week for 10 weeks.

Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L.

Eye examinations are conducted at months 1, 3, 6, 9, and 12. These examinations consist of BSCVA, NEI-VFQ and laser Doppler measurement of blood flow. Improvement of one line in BSCVA is observed at one and three months, but not at six months. Consequently, four additional infusions of P188 are given at weekly intervals. The BSCVA again improves one line and remains stable through 12 months. In addition, the ChBFlow measured by laser Doppler flowmetry stabilized at a level 2.2 AU above the baseline value of 6.1 AU. It is significant that vision does not deteriorate during the year as expected in patients with Category 4 AMD. Vision is maintained in a patient at high risk of rapid deterioration over a period of months.

### EXAMPLE 5

### Use of Poloxamer 188 to Treat Age-Related Macular Degeneration

Poloxamer 188 is administered to a 77 year old man with Age-Related Macular Degeneration with multiple large soft drusen near the foveal center of his left eye. He has no cataract or other retinal disease. His eyes have a best spectacle corrected visual acuity (BSCVA) using the Early Treatment Diabetic Retinopathy Study (ETDRS) chart of 20/50 on the left and 20/40 on the right. His kidney function is normal for his age.

This patient is treated with an intravenous infusion of purified Poloxamer 188 at a constant rate over 24 hours to a total dose of 800 mg/kg body weight. Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L. It is mixed with either normal saline of 5% dextrose to produce a volume convenient for the infusion apparatus. and at the end of the infusion. ChBFlow, measured by laser Doppler flowmetry improves from 5.7 AU to 7.0 AU at the end of infusion. Edema improves within 3 days measured by reduction of mean retinal thickness measured by optical coherence tomography.

### EXAMPLE 6

### Use of Poloxamer 188 to Treat Age-Related Macular Degeneration

A 75-year-old woman with vision loss in her right eye for two years and recent gradual loss of vision in her left eye is diagnosed with bilateral neovascular AMD with occult choroidal neovascularization. Her visual acuity (VA) is counting fingers (CF) at six feet in her right eye and 20/80 in her left eye. The patient receives intravitreal injection of pegaptanib (MACUGEN®, a pegylated anti-VEGF aptamer) to both eyes, but her vision drops to CF at six feet in the right eye and 20/100 in the left eye. Optical coherence tomography reveals increasing fluid and cystic maculopathy in both eyes.

This patient is treated with an intravenous infusion of purified Poloxamer 188 at a constant rate of 30 mg/gk/hour for eight hours on five successive days and weekly for four weeks more. Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L. It is mixed with either normal saline of 5% dextrose to produce a volume convenient for the infusion apparatus. Fundus examination shows resolution of subretinal fluid in both eyes, and this is confirmed by optical coherence tomography and laser Doppler measurement of blood flow. Her visual function is improved in the left eye to 20/60.

### EXAMPLE 7

### Use of Poloxamer 188 in Combination with LUCENTIS® to Treat Wet Age-Related Macular Degeneration

A 82-year-old man with vision loss in both eyes for two years is diagnosed with bilateral neovascular AMD with predominantly classic choroidal neovascularization. His visual acuity is counting fingers at six feet in his left eye and 20/100 in his right eye. Optical coherence tomography reveals increasing fluid in the retina of both eyes.

LUCENTIS® (ranibizumab; an anti-VEGF monoclonal antibody fragment) 0.5 mg (0.05 mL) is administered into the left eye by intravitreal injection once a month for 4 months and thereafter once every 3 months. This patient is simultaneously treated with an intravenous infusion of purified Poloxamer 188 at a constant rate of 100 mg/kg/hour over four hours. The is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L. It is mixed with either normal saline of 5% dextrose to produce a volume convenient for the infusion apparatus.

Fundus examination shows resolution of subretinal fluid in both eyes. This is confirmed by optical coherence tomography. The vision demonstrates marginal improvement within one month and remains stable for one year.

### EXAMPLE 8

### Use of Poloxamer 188 to Treat Age-Related Macular Degeneration

A 81-year-old man with vision loss in his left eye for two years and recent gradual loss of vision in his right eye is diagnosed with bilateral neovascular AMD with predominantly classic choroidal neovascularization. His visual acuity is counting fingers at six feet in his left eye and 20/80 in his right eye. Optical coherence tomography reveals increasing fluid in the retina of both eyes.

This patient is treated with an intravenous infusion of purified Poloxamer 188 at a constant rate of 100 mg/kg/hour over four hours. The infusion are repeated twice on the first week and weekly thereafter for four weeks. Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L. It is mixed with either normal saline of 5% dextrose to produce a volume convenient for the infusion apparatus.

Fundus examination shows resolution of subretinal fluid in both eyes. This is confirmed by optical coherence tomography. The vision demonstrates improvement within one week and remains stable for one year.

### EXAMPLE 9

### Use of Poloxamer 188 to Treat Peripheral Arterial Disease

A 65 year old man with Fontaine state III peripheral arterial disease in both legs that has caused exercise-limiting symptoms for several months is treated with an intravenous infusion of purified Poloxamer 188 at a constant rate over two hours to a total dose of 100 mg/kg body weight. This treatment is repeated weekly for 10 weeks.

Poloxamer 188 is formulated as 15 g of purified Poloxamer 188 (150 mg/ml), 308 mg sodium chloride USP, 238 mg sodium citrate USP, 36.6 mg citric acid USP and Water for 312 mOsm/L.

Transcutaneous oxygen pressure (TcpO₂) (PeriFlux System 500, Perimed, Stockholm Sweden) and tissue oxygen saturation by near infrared spectroscopy (InSpectra StO₂, Hutchinson Technologies, Hutchinson Minn.) are repeatedly measured in both legs to monitor the effects of treatment on the microcirculation. The measurements at 1, 3, 6, and 12 months after beginning of treatment show an improvement of Fontaine stage, a pronounced increase in TcpO₂, and regression of the rest pain.

### EXAMPLE 10

### Use of Poloxamer 188 to Treat Critical Limb Ischemia

Poloxamer 188 is administered to a 78 year old woman who has peripheral vascular disease that has progressed to Fontaine stage III critical limb ischemia with persistently recurring rest pain for three weeks and foot ulceration. Her TcpO₂ is 39 mmHg, which identifies her as high risk for amputation. She is treated with an intravenous infusion of purified Poloxamer 188 at a constant rate over four hours to a total dose of 200 mg/kg body weight. This treatment is repeated daily for four days and then weekly for ten weeks.

Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L.

Rest pain and foot ulceration improve. In addition, the transcutaneous oxygen pressure (TcpO₂) and ankle-brachial index (ABI) are repeatedly determined. The TcpO₂ improves and amputation is avoided.

### EXAMPLE 11

### Use of Poloxamer 188 to Treat Macular Edema of Diabetic Retinopathy

A 44 year old woman with blurred vision due to macular edema of diabetic retinopathy is treated with laser surgery and with an intravenous infusion of purified Poloxamer 188 at a constant rate over four hours to a total dose of 100 mg/kg body weight. This treatment is repeated weekly for six weeks.

Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of and vision stabilizes.

### EXAMPLE 12

### Use of Poloxamer 188 to Treat Proliferative Diabetic Retinopathy

A diabetic 40 year old man with recurrent proliferative retinopathy, who had previously been subjected to retinal laser surgery, is treated with an intravenous infusion of purified Poloxamer 188 at a constant rate over four hours to a total dose of 100 mg/kg body weight. This treatment is repeated weekly for eight weeks.

Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L.

Edema of the retinal blood vessels is monitored by fluorescein angiography optical coherence tomography and laser Doppler flowmetry. If improvement is observed, additional laser surgery to ablate vessels will not be required.

### EXAMPLE 13

### Use of Poloxamer 188 to Treat Transient Ischemic Attacks

A 63 year old man suffering from increasing transient ischemic attacks characterized by hemiparesis is treated with an intravenous infusion of purified Poloxamer 188 at a constant rate over four hours to a total dose of 200 mg/kg body weight. This treatment is repeated weekly for eight weeks with a lower dose of 100 mg/kg.

Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L.

The incidence and severity of transient ischemic attacks diminishes during the first week and remains low to absent for three months.

### EXAMPLE 14

### Use of Poloxamer 188 to Treat Chronic Congestive Heart Failure with Unstable Angina

A 72 year old man with a two-year history of congestive heart failure suffers an acute episode with unstable angina pectoris. He is given standard therapy of digoxin, diuretics and supportive measures. In addition, he is infused intravenously with purified Poloxamer 188 at a daily for three days and then weekly for eight weeks with a lower dose of 100 mg/kg.

Poloxamer 188 is formulated as 15 g of purified Poloxamer 188 (150 mg/ml), 308 mg sodium chloride USP, 238 mg sodium citrate USP, 36.6 mg citric acid USP and Water for Injection USP Qs to 100 ml. The pH of the solution is approximately 6.0 and has an osmolarity of 312 mOsm/L.

The unstable angina and the heart failure are monitored for improvement. The episodes of unstable angina diminish and cease within the first week and do not recur for at least three months.

All scientific articles, publications, abstracts, patents and patent applications mentioned herein are herebyincorporated by reference in their entireties.

While this invention has been described in specific detail with reference to the disclosed embodiments, it will be understood that many variations and modifications may be effected within the spirit and scope of the invention.

1. A method of treating a chronic microvascular disease in a mammal, comprising administering to the mammal a pharmaceutical composition comprising a pharmaceutically effective amount of a polyoxyethylene/polyoxypropylene copolymer and a pharmaceutically acceptable carrier, wherein the copolymer has the following chemical formula:
HO(C₂H₄O)ₐ—(C₃H₆O)_{b}—(C₂H₄O)ₐH
that can also be written as 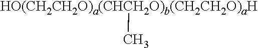
wherein (C₃H₆O)_{b} is a hydrophobe portion of the copolymer and wherein (C₂H₄O)ₐ is a hydrophile portion of the copolymer,
wherein b is an integer such that the hydrophobe portion represented by (C₃H₆O)_{b} has a molecular weight of approximately 950 to 4000, and wherein a is an integer such that the hydrophile portion represented by (C₂H₄O)ₐ constitutes approximately 50% to 90% by weight of the copolymer,
thereby treating the chronic microvascular disease in the mammal.

2. The method of claim 1, wherein b is an integer such that the hydrophobe portion of the copolymer represented by (C₃H₆O)_{b} has a molecular weight of approximately 1200 to 3500, and wherein a is an integer such that the hydrophile portion represented by (C₂H₄O)ₐ constitutes approximately 50% to 90% by weight of the copolymer.

3. The method of claim 1, wherein the molecular weight of the hydrophobe portion represented by (C₃H₆O)_{b} is approximately 1750 daltons and wherein the total molecular weight of the copolymer is approximately 8400 daltons.

4. The method of claim 1, wherein the chronic microvascular disease to be treated is characterized by inflammation.

5. The method of claim 1, wherein the chronic microvascular disease to be treated is selected from the group consisting of: age-associated macular degeneration, diabetic retinopathy, diabetic peripheral vascular disease, sudden hearing loss, cerebral ischemia, transient ischemic attacks, congestive heart failure, critical limb ischemia, and peripheral vascular disease.

6. The method of claim 1, wherein the administration is via intravenous infusion.

7. The method of claim 1, wherein a single administration of the pharmaceutical composition achieves a desired effect.

8. The method of claim 1, wherein the pharmaceutical composition further comprises at least one of: an anti-VEGF compound, an antithrombotic compound, or an anti-inflammatory compound.

9. The method of claim 3, wherein the polyoxyethylene/polyoxypropylene copolymer is purified to reduce low and high molecular weight contaminants such that the copolymer has a polydispersity value of less than approximately 1.07.

10. The method of claim 9, wherein the copolymer has a polydispersity value of less than approximately 1.05.

11. The method of claim 10, wherein the copolymer has a polydispersity value of less than approximately 1.03.

12. A method of diagnosing a chronic microvascular disease in a mammal comprising:
(a) administering to the mammal a pharmaceutical composition comprising a pharmaceutically effective amount of a polyoxyethylene/polyoxypropylene copolymer and a pharmaceutically acceptable carrier, wherein the copolymer has the following chemical formula:
HO(C₂H₄O)ₐ—(C₃H₆O)_{b}—(C₂H₄O)ₐH
that can also be written as 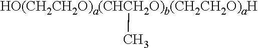
wherein (C₃H₆O)_{b} is a hydrophobe portion of the copolymer and wherein (C₂H₄O)ₐ is a hydrophile portion of the copolymer,
wherein b is an integer such that the hydrophobe portion represented by (C₃H₆O)_{b} has a molecular weight of approximately 950 to 4000, and wherein a is an integer such that the hydrophile portion represented by (C₂H₄O)ₐ constitutes approximately 50% to 90% by weight of the copolymer,
(b) measuring an amount of an inflammatory indicator in a bodily fluid of the mammal, and
(c) correlating a change in the amount of the inflammatory indicator with the identity of a chronic microvascular disease,
thereby diagnosing a chronic microvascular disease in the mammal.

13. The method of claim 12, wherein b is an integer such that the hydrophobe portion of the copolymer represented by (C₃H₆O)_{b} has a molecular weight of approximately 1200 to 3500 and wherein a is an integer such that the hydrophile portion represented by (C₂H₄O)ₐ constitutes approximately 50% to 90% by weight of the copolymer.

14. The method of claim 12, wherein the molecular weight of the hydrophobe (C₃H₆O)_{b} is approximately 1750 daltons and wherein the total molecular weight of the copolymer is approximately 8400 daltons.

15. The method of claim 14, wherein the polyoxyethylene/polyoxypropylene copolymer is purified to reduce low and high molecular weight contaminants such that the copolymer has a polydispersity value of less than approximately 1.07.

16. The method of claim 15, wherein the copolymer has a polydispersity value of less than approximately 1.05.

17. The method of claim 16, wherein the copolymer has a polydispersity value of less than approximately 1.03.

18. The method of claim 12, wherein the inflammatory indicator is secretory phospholipase A₂.

19. The method of claim 12, wherein the inflammatory indicator is selected from the group consisting of: erythrocyte sedimentation rate, Factor B, fibrinogen, C-reactive protein, IL-1, IL-6, IL-8, and monocyte chemotractant protein-1.

20. The method of claim 12, wherein the pharmaceutical composition further comprises an anti-VEGF compound or an anti-inflammatory compound.
